(19) United States
(12) Patent Application Publication
(10) Pub. No.: US 2014/0276520 A1
(43) Pub. Date: Sep. 18, 2014

(54) HYPERBOLIC SANITARY PAD SYSTEM

(71) Applicant: Linda Siegel, Sarasota, FL (US)

(72) Inventor: Linda Siegel, Sarasota, FL (US)

(21) Appl. No.: 13/843,394

(22) Filed: Mar. 15, 2013

(51) Int. Cl. A61F 13/47

(52) U.S. Cl. CPC A61F 13/4704
USPC 604/385.01

(57) ABSTRACT

The present invention provides a hyperbolic sanitary pad system including an absorbent elongated body including a first and second curved surfaces extending from a substantially planar surface the elongated body extending between a first and second parabolic end and a crest located along a central axis positioned along a radius curvature between the pair of curved side surfaces and a support surface further comprising a pair of downwardly depending lips, presenting an elliptical cavity therebetween, the first and second parabolic ends angularly extending from the elongated body for securing fluids.

### RELATED APPLICATION

The present provisional patent application is a provisional patent application based upon the non-provisional patent application No. 61/730,491 filed Nov. 27, 2012 which was based upon the non-provisional application Ser. No. 13/461,138 filed May 1, 2012, which is a continuation in part from Ser. No. 12/456,957 which issued as a patent on May 1, 2012 the subject matter of which is incorporated herein by reference.

### FIELD OF THE INVENTION

The present invention relates to sanitary pad system and more particularly pertains to a hyperbolic shaped pad for securing bodily fluid while maintaining the pad in a proper absorbing position through a projecting crest, the securing being effected in a comfortable reliable, hygienic, sanitary, convenience and economical manner.

### BACKGROUND OF THE INVENTION

The use of sanitary pads of known designs and configurations is known in the prior art. More specifically, sanitary napkin, sanitary towel, sanitary pad, menstrual pad, maxi pad, or pad are generally known and used as an absorbent item worn by a woman while having bladder or incontinence issues or while she is menstruating, recovering from vaginal surgery, for lochia (post birth bleeding), abortion, or any other situation where it is necessary to absorb a flow of blood from a woman's vagina.

These pads are different from higher absorbency incontinence pads, which are worn by both men and women who have urinary incontinence problems or experience stress incontinence. Menstrual pads and incontinence pads are different from each other, where the menstrual pad is typically worn by women during menstruation with more absorbency, the incontinence pad having lower absorbency which may be worn by either a man or women, but not during menstruation.

The ergonomic design and materials used to make sanitary pads has also evolved through the years. Traditionally the sanitary pad was not designed to be ergonomic. In some cases, an external belt is utilized and in other cases, towels and mud have been utilized. Overall, early attempts have not been absorbent and effective as current products. In fact, early pads were up to an inch thick with leaks being a major problem. Some changes to the contour include quilting of the lining and adding “wings” along the side of the pad. In addition, the pad thickness has been reduced by utilizing superabsorbent materials such as sphagnum and polyacrylate—superabsorbent gels derived from petroleum within the pad. However, most materials used in the manufacture of pads utilize non-ecofriendly resources, instead depending upon petroleum and forestry products for their fabrication. For example, the absorbent core, which was originally made from chlorine bleached wood pulp, has been combined with polyacrylate gels to reduce the pads thickness while helping to absorb any fluid quickly and retaining the fluid in a suspension. The cover stock is non-woven polypropylene and the leak-proof barrier is made from a polyethylene film.

By way of example, U.S. Pat. No. 7,217,259 issued May 15, 2007 to McDaniel relates to an Interlabial Absorbent Article. U.S. Pat. No. 5,702,380 issued Dec. 30, 1997 to Walker relates to a Sanitary Napkin. Lastly, U.S. Pat. No. 5,672,165 issued Sep. 30, 1997 to Belecky relates to a Menstrual Hygiene Product.

These devices do not describe a sanitary pad system that allows for securing bodily fluid pads in the illustrated absorbing position, the pad being minimally designed and secured in an anatomical, comfortable, reliable, hygienic, sanitary, convenient and economical manner.

### SUMMARY OF THE INVENTION

In view of the foregoing disadvantages inherent in the known types of sanitary pads of known designs and configurations now present in the prior art, the present invention provides an improved hyperbolic sanitary pad system with a parabolic absorption surface extending longitudinally from a pair of outwardly angled parabolic protrusions and laterally between a pair of inflection axes presenting an upwardly extending crest and an elliptical cavity formed between a pair of downwardly depending lips, the lips being adapted for proper placement during use.

In a stored embodiment, the body portion prior to use has a planar surface with a vertically oriented projection extending upwardly from the planar body surface. The projection extends longitudinally along a longitudinal axis extending along a central area of the body portion. The projection extends between an upper vertical position and a lower body portion near the planar body surface.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved panty liner/pad system which includes at least some of the advantages of the prior art sanitary pads of known designs and configurations with the additional benefits provided by the present design.

It is another object of the present invention to provide a new and improved panty liner/pad system which may be easily and efficiently manufactured and marketed.

It is further object of the present invention to provide a new and improved panty liner/pad system which is of durable and reliable constructions.

An even further object of the present invention is to provide a new and improved sanitary pad system which is susceptible of a low cost of manufacture with regard to both materials and labor, which takes up less landfill space, has less waste and which accordingly is then susceptible of low prices of sale to the consuming public, thereby making such sanitary pad system economically available to the buying public.

Even still another object of the present invention is to provide a hyperbolic sanitary pad system for securing bodily fluid pads in a proper absorbing position through a projecting ridge peak, the securing being effected in a comfortable, reliable, hygienic, sanitary, convenient and economical manner.

Lastly, it is an object of the present invention to provide a new and improved sanitary pad system with a central body portion having triangular ends spaced centrally along the longitudinal axis by a length and oppositely curved side surfaces where the sides are spaced by a minimum width at a crest extending vertically away from the generally planar lower surface.

Generally, the elongated body portion has a parabolic upper surface and generally planar lower surface. The vertically oriented crest extends upwardly from a pair of downwardly depending lips. The elongated body portion also has a support structure for securely positioning the improved sanitary pad system during use for capturing any body fluids.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is an upper side perspective view of the improved hyperbolic sanitary pad system in accordance with one embodiment of the present invention.

FIG. 2 is a bottom rear perspective view of the improved hyperbolic sanitary pad system of FIG. 1.

FIG. 3 is a bottom plan of the improved hyperbolic sanitary pad system of FIG. 1.

FIG. 4 is a side elevation view of the improved hyperbolic sanitary pad system of FIG. 1.

FIG. 5 is top plane view of the improved hyperbolic sanitary pad system of FIG. 1.

FIG. 6 is an end view taken along line A-A of FIG. 4

FIG. 7 is a front view taken along line A-A of FIG. 4

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

With reference now to the drawings, and in particular to FIG. 1 thereof, the preferred embodiment of the new and improved hyperbolic sanitary pad system embodying the principles and concepts of the present invention and generally designated by the reference numeral 10.

The present invention, the hyperbolic sanitary pad system 10 is comprised of a plurality of components. Such components in their broadest context include an absorbent elongated body 20 extending longitudinally between a pair of angled projections 30 and a vertical structure 40 extending from a pair of depending lips 42 to a crest 44. Such components are individually configured and correlated with respect to each.

The pair of triangular ends with rounded tops 30 are spaced longitudinally by a length sufficient to catch body fluids, but in some embodiments, may be shortened to sufficiently contain solid waste as well as other body fluids as may be necessary in senior male or female users. The angled triangular ends 30 as shown extend from the generally planar lower surface 26 with each side corresponding to one of the pair of curved side surfaces 32 extending longitudinally along the longitudinal axis. The curved side surfaces 32 extend between the support structure 50 and the crest 44 along the longitudinal axis.

The support structure 50 includes the downwardly depending lips 42 and an elongated flange which extend longitudinally along the elongated body 20. The support structure 50 generally presents and ergonomic contour and is adapted for outer skin contact with the user while helping to retain the shape of the pad 10 during use. The downwardly depending lips 42 may be fabricated from a moisture impervious material and may further include a covering material (not shown). During use, the covering material may be used in connection with an article of clothing or may be used to secure the downward depending lips to each other.

The absorbent elongated body 20 may be fabricated from a fibrous, wicking and absorbent material which may also provide odor absorbing properties. In one embodiment, the absorbent material may include Coir or other natural fibers. The downwardly depending lips 42 extend longitudinally from an elongated flange which is connected to the curved side surfaces 32. An elliptical cavity 34 spanning the downwardly depending lips 42 and extending longitudinally along the planar surface 26, opposite the elongated body 20.

The curved sides 32, corresponding to pair of inflection axis extending longitudinally along the elongated body 20, extend from each of the pair of downwardly depending lips 44 towards the crest 44. The curved sides 32 are spaced centrally along the elongated body 20 by a width. The elongated body 20 has a longitudinal axis which extends between the pair of triangular ends 30 and a latitudinal axis which extends between the pair of curved surfaces 32.

Prior to use, the body portion includes a generally planar surface 26 with the support structure 50 extending latitudinal from the elongated body 20 in a stored configuration. To use the pad, the user may remove any covering 56 extending longitudinally along the underside of the downwardly depending lips 42.

The crest 44 is presented by the pair of curved side surfaces 32 and generally extends vertically from the generally planar surface 26 a height. The crest 44 is the upper limit of the elongated body 20 and is adapted for receipt within the desired contour which depending on whether the user is a man or woman, may be a specific area.

The planar surface 26 adjoins the elongated body 20 opposite the crest 44, the supporting structure 50 elongated along the longitudinal axis. The planar surface 26 is centrally positioned along the longitudinal axis of the elongated body 20 for placement within a specific area. The crest 44 has an upper edge which is centrally positioned along the longitudinal axis of the elongated body 20 and rises vertically a peak distance above the main portion of the elongated body 20.

The elongated body 20 extends between a pair of triangular ends 30 being offset an angle from the elongated body 20 presenting a leading 30a and trailing edge 30b, respectively associated with the first and second triangular ends 30. The inner region of the pair of triangular ends being associated with the an optionally formed elliptical cavity 34 while the outer region of the pair of triangular ends is associated with the crest 44.

Further provided is a sheet 38 of a moisture impervious material in association with the underside of the lower planar surface 26 which may or may not have stitching for attaching to the lower surface of the elongated body 20.

Optionally, a covering may be provided which, if provided, may be removed prior to exposing each depending lip 42. The covering (not shown) may be positioned parallel with and laterally spaced from the longitudinal axes and adapted for removable use. In operation, the support structure 50 is adapted for placement near a user's under garments concurrently with the receipt of the crest 44 within an ergonomic contour.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Having thus described the invention, what is claimed as new and desired to be secured by Letters Patent is as follows:

1. A hyperbolic sanitary pad system comprising:
an absorbent elongated body said elongated body being parabolic and including a first and second curved surfaces extending from a substantially planar surface, said elongated body extending between a first and a second parabolic end;
a crest located along a central axis positioned along a radius curvature between said pair of curved side surfaces;
a support surface further comprising a pair of downwardly depending lips and presenting an elliptical cavity therebetween, and
said first and second parabolic ends angularly extending from said elongated body.

2. The hyperbolic sanitary pad system of claim 1 further comprising a vertical structure extending from said pair of downwardly depending lips to said crest.

3. The hyperbolic sanitary pad system of claim 2 wherein said elongated body extends from said

4. The hyperbolic sanitary pad system of claim 1 wherein said support surface further presents an ergonomic contour.

5. The hyperbolic sanitary pad system of claim 1 wherein said support structure further comprises an elongated flange.

6. The hyperbolic sanitary pad system of claim 1 wherein said downwardly depending lips comprises a moisture impervious material.

7. The hyperbolic sanitary pad system of claim 1 wherein said elliptical cavity extends longitudinally along said planar surface.

8. The hyperbolic sanitary pad system of claim 1 wherein said elongated body is substantially parabolic.

9. The hyperbolic sanitary pad system of claim 1 wherein said radius curvature is small.

10. The hyperbolic sanitary pad system of claim 1 wherein said curved side surfaces correspond to a pair of inflection axis extending longitudinally along said elongated body.

11. The hyperbolic sanitary pad system of claim 1 wherein said elongated body has a longitudinal axis which extends between a pair of triangular ends.

12. The hyperbolic sanitary pad system of claim 1 wherein said elongated body has a latitudinal axis which extends between the pair of curved surfaces.
